(19) United States
(12) Patent Application Publication
(10) Pub. No.: US 2003/0207277 A1
(43) Pub. Date: Nov. 6, 2003

(54) Human deoxyguanosine kinase (dGK) variants associated with cancers

(76) Inventor: Ken-Shwo Dai, Hsinchu (TW)

Correspondence Address: Ladas & Parry, 26 West 61st Street, New York, NY, 10023, US

(21) Appl. No.: 10/103,959

(22) Filed: Mar. 22, 2002

(51) Int. Cl. C12Q 1/68; C07H 21/04; C12P 19/34

(52) U.S. Cl. 435/6; 435/91.2; 536/23.1

(57) ABSTRACT

The invention relates to the nucleic acid sequences of two novel human dGK-related variants (dGK1 and dGK2).

The invention further relates to the use of the nucleic acid sequences of the variants in diagnosing diseases associated with the deficiency of dGK gene, in particular, cancers, e.g. uterus and placenta cancers.

### FIELD OF THE INVENTION

The invention relates to the nucleic acid of novel human dGK variants, and the uses of the same in diagnosing diseases associated with the deficiency of dGK gene, in particular, cancers, e.g. uterus cancer or placenta cancer.

### BACKGROUND OF THE INVENTION

Cancers are the major causers of deaths in the world. In recent years, much progress has been made toward understanding the molecular and cellular biology of cancers. Many important contributions have been made by the identification of several key genetic factors associated with cancers. However, the treatments of cancers still mainly depend on surgery, chemotherapy, and radiotherapy. This is because the molecular mechanisms underlying the pathogenesis of cancers remain largely unclear. It has been shown that the balance between proliferation and apoptosis (a process of programmed cell death) in normal cells was disrupted in cancer cells (Reed, (1999) J Clin Oncol 17:2941-53; Sjostrom and Bergh, (2001) BMJ 322:1538-9). Great interest was recently raised in understanding the role of apoptosis in cancers (Wyllie et al. (1999) Br J Cancer 80 Suppl 1:34-7; Sjostrom and Bergh, (2001) BMJ 322:1538-9; Zornig et al. (2001) Biochim Biophys Acta 1551:F1-37). Therefore, future strategies for the prevention and treatment of cancers will be focused on the elucidation of these genetic substrates, in particular, the genes associated apoptosis.

One of the characteristics of apoptosis is the DNA fragmentation. It is known that a balanced supply of deoxynucleotide triphosphate (dNTP) is crucial to the fidelity of DNA synthesis and repair. A previous report indicated that dNTP concentrations were approximately 3-fold increased in patients with leukaemia and other myeloproliferative diseases (Tattersall et al. (1980) Antibiot Chemother 28:94-101). An imbalance in the availability of dNTP preceding the DNA fragmentation suggests that the factors associated with alterations in dNTP supply are crucial to the fragmentation of DNA in apoptotic cells (Oliver et al. (1996) Experientia 52:995-1000). The observation of a suppression of transcriptional down-regulation of genes, encoding enzymes responsible for the DNA precursor synthesis, by DNA tumor viruses (Hengstschlager et al. (1994a) Cell Growth Differ 5:1389-94; Hengstschlager et al. (1994b) J Biol Chem 269:13836-42) supports the role of dNTP synthesis pathways in the pathogenesis of cancers. Two pathways (the de novo pathway and the salvage pathway) are known to be involved in the synthesis of dNTP precursors. The salvage pathway has been shown to be associated with the dNTP pool balance in apoptotic cells (Oliver et al. (1996) Experientia 52:995-1000). Four enzymes involved in the salvage pathway are deoxycytidine kinase, thymidine kinase 1 and 2 and deoxyguanosine kinase (Arner and Eriksson, (1995) Pharmacol Ther 67:155-86). Deoxyguanosine kinase (dGK) was reported to be involved in the apoptotic process (Jullig M, Eriksson (2001) J Biol Chem 276:24000-4). Furthermore, the assignment of dGK to chromosome 2p (Johansson et al. (1996) Genomics 1996 38:450-1), which is a region of chromosomal abnormalities associated with many cancers (Merlo et al. (1994) Cancer Res 54:2098-101; Otsuka et al. (1996) Genes Chromosomes Cancer 16:113-9; Feder et al. (1998) Cancer Genet Cytogenet 102:25-31; Jones et al. (2001) Am J Pathol 158:207-14; Lui et al. (2001) Int J Oncol 19:451-7; Summersgill et al. (2001) Br J Cancer 85:213-20; Willatt et al. (2001) Am J Med Genet 102:304-5), strengthened the role of dGK in the pathogenesis of cancers. It is thus believed that dGK and/or its gene variants are associated with mechanisms of carcinogenesis.

### SUMMARY OF THE INVENTION

The present invention provides the nucleic acid sequences of two dGK-related gene variants (dGK1 and dGK2) and the fragments thereof. The nucleic acid sequences of these variants can be used for the diagnosis of diseases associated with the deficiency of dGK gene, in particular, cancers, e.g. uterus cancer or placenta cancer.

The invention also provides methods for diagnosing diseases associated with deficiency of dGK gene, in particular, cancers, e.g. uterus cancer or placenta cancer.

### BRIEF DESCRIPTION OF THE DRAWINGS

FIG. 1 shows the nucleic acid sequence (SEQ ID NO: 1) and amino acid sequence (SEQ ID NO:2) of dGK1.

FIG. 2 shows the nucleic acid sequence (SEQ ID NO:3) and amino acid sequence (SEQ ID NO:4) of dGK2.

FIGS. 3A-G show the nucleotide sequence alignment between the human dGK gene and its variants (dGK1 and dGK2).

FIGS. 4A-B shows the amino acid sequence alignment between the human dGK protein and its variants (dGK1 and dGK2).

### DETAILED DESCRIPTION OF THE INVENTION

According to the present invention, all technical and scientific terms used have the same meanings as commonly understood by persons skilled in the art.

The term “base pair (bp)” used herein denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402)” used herein denotes programs for evaluation of homologies between a query sequence (amino or nucleic acid) and a test sequence as described by Altschul et al. (Nucleic Acids Res. 25: 3389-3402, 1997). Specific BLAST programs are described as follows:

(1) BLASTN compares a nucleotide query sequence with a nucleotide sequence database;

(2) BLASTP compares an amino acid query sequence with a protein sequence database;

(3) BLASTX compares the six-frame conceptual translation products of a query nucleotide sequence with a protein sequence database;

(4) TBLASTN compares a query protein sequence with a nucleotide sequence database translated in all six reading frames; and

(5) TBLASTX compares the six-frame translations of a nucleotide query sequence with the six-frame translations of a nucleotide sequence database.

The term “cDNA” used herein denotes nucleic acids synthesized from a mRNA template using reverse transcriptase.

The term “cDNA library” used herein denotes a library composed of complementary DNAs which are reverse-transcribed from mRNAs.

The term “complement” used herein denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion” used herein denotes a removal of a portion of one or more amino acid residues/nucleotides from a gene.

The term “expressed sequence tags (ESTs)” used herein denotes short (200 to 500 base pairs) nucleotide sequences derived from either 5′ or 3′ end of a cDNA.

The term “in silico” used herein denotes a process of using computational methods (e.g., BLAST) to analyze DNA sequences.

The term “polymerase chain reaction (PCR) used herein denotes a method which increases the copy number of a nucleic acid sequence using a DNA polymerase and a set of primers (about 20 bp oligonucleotides complementary to each strand of DNA) under suitable conditions (successive rounds of primer annealing, strand elongation, and dissociation).

The term “protein” or “polypeptide” used herein denotes a sequence of amino acids in a specific order that can be encoded by a gene or by a recombinant DNA. It can also be chemically synthesized.

The term “nucleic acid sequence” or “polynucleotide” used herein denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR)” used herein denotes a process which transcribes mRNA to complementary DNA strand using reverse transcriptase followed by polymerase chain reaction to amplify the specific fragment of DNA sequences.

The term “transformation” used herein denotes a process describing the uptake, incorporation, and expression of exogenous DNA by prokaryotic host cells.

The term “transfection” used herein denotes a process describing the uptake, incorporation, and expression of exogenous DNA by eukaryotic host cells.

The term “variant” used herein denotes a fragment of sequence (nucleotide or amino acid) inserted or deleted by one or more nucleotides/amino acids.

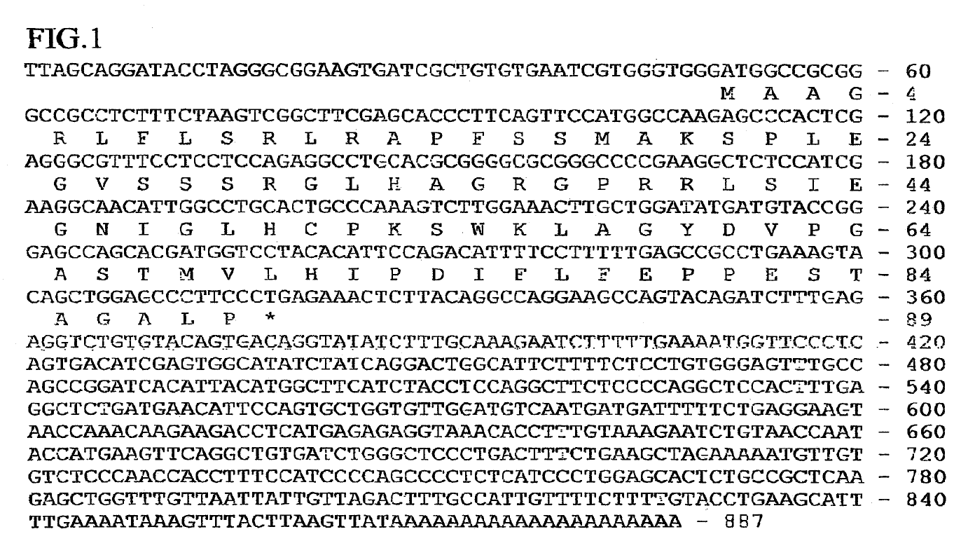

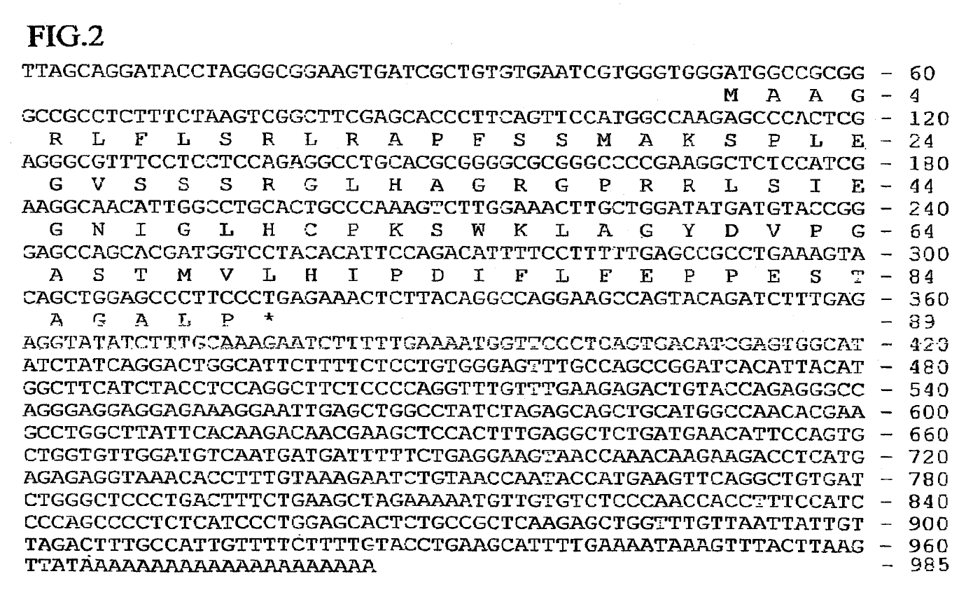

The present invention provides the nucleic acid sequences of the novel human dGK variants (dGK1 and dGK2) and the fragments thereof. According to the present invention, human dGK cDNA sequence was used to query the human EST databases (pooled cancers) using BLAST program to search for dGK-related gene variants. Two human cDNA partial sequences (i.e., ESTs) deposited in the databases showing similarity to dGK were isolated and sequenced. These clones were isolated from a cDNA library constructed using pooled cancer tissues. FIG. 1 shows the nucleic acid sequence of dGK1 (SEQ ID NO: 1) and its corresponding amino acid sequence encoded thereby (SEQ ID NO: 2). The full-length of the dGK1 cDNA clone is 887 bp containing an open reading frame (ORF) of 267 bp extending from nucleotides 51 to 317, which corresponds to an encoded protein of 89 amino acid residues with a predicted molecular mass of 9.4 kDa. FIG. 2 shows the nucleic acid sequence of dGK2 (SEQ ID NO: 3) and its corresponding amino acid sequence encoded thereby (SEQ ID NO: 4). The full-length of the dGK2 cDNA clone is 985 bp containing an open reading frame (ORF) of 267 bp extending from nucleotides 51 to 317, which corresponds to an encoded protein of 89 amino acid residues with a predicted molecular mass of 9.4 kDa. It should be noted that both dGK1 and dGK2 have the same predicted peptide sequence. The sequence around the initiation ATG codon of both dGK1 and dGK2 (located at nucleotides 51 to 53) was similar to the Kozak consensus sequence (A/GCCATGG) (Kozak, (1987) Nucleic Acids Res. 15: 8125-48; Kozak, (1991) J Cell Biol. 115: 887-903.).

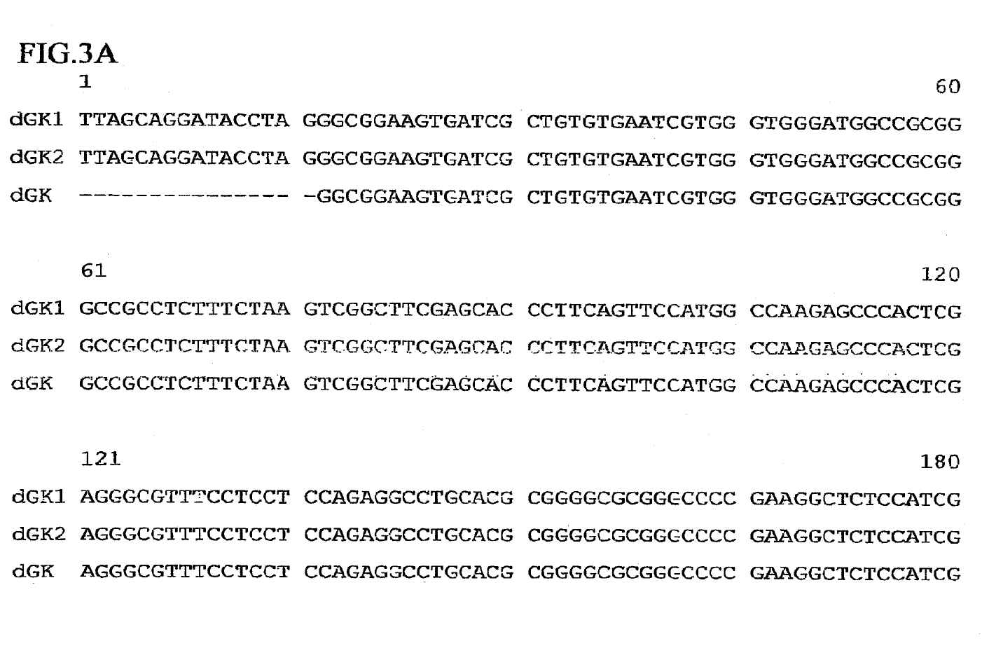

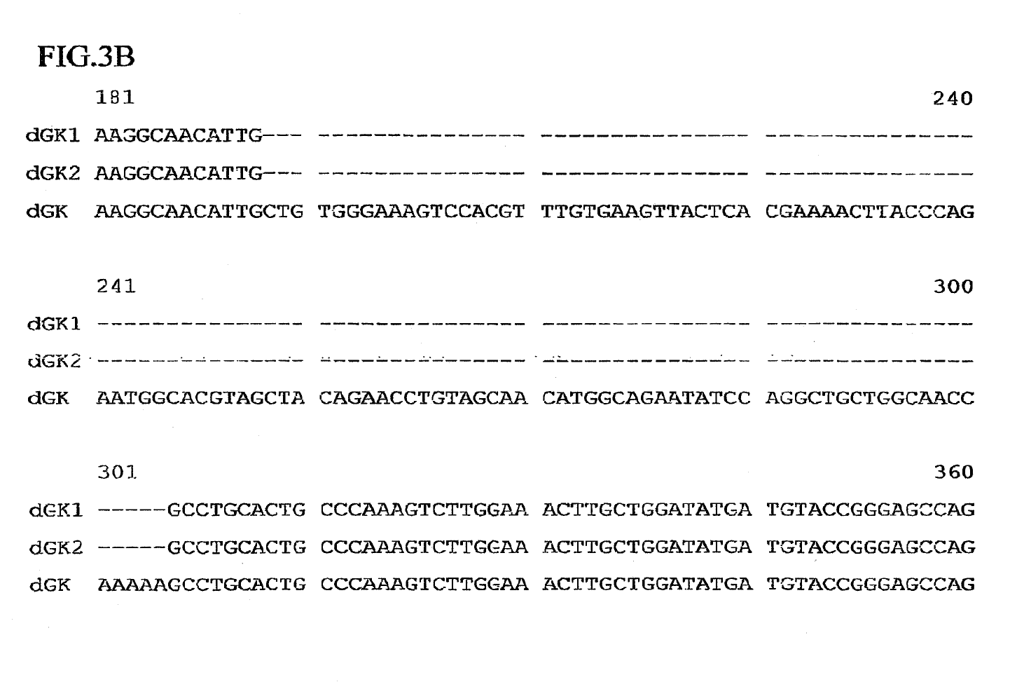

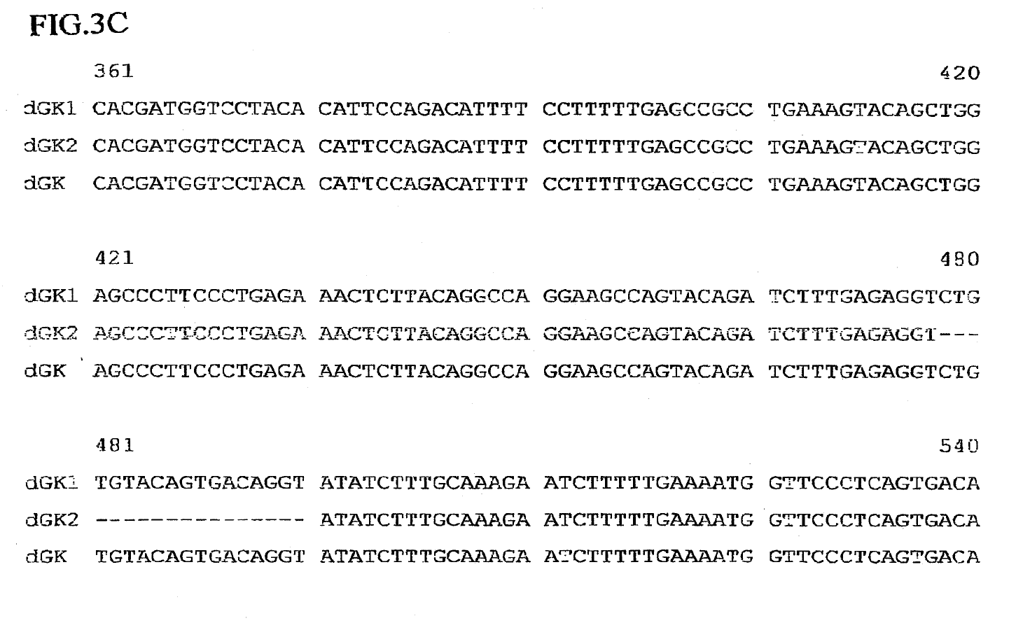

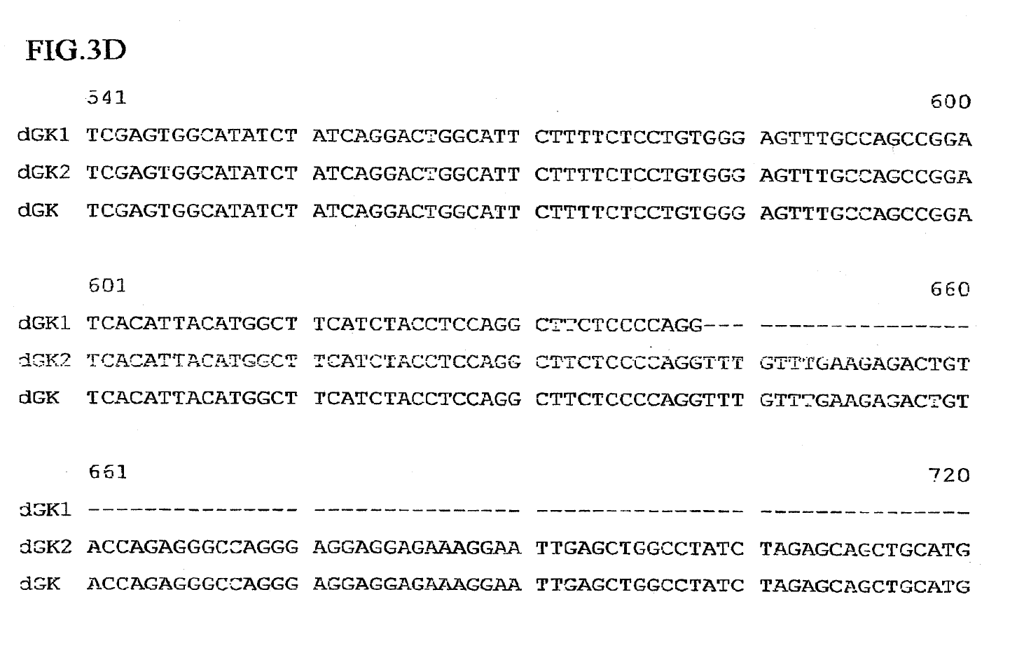

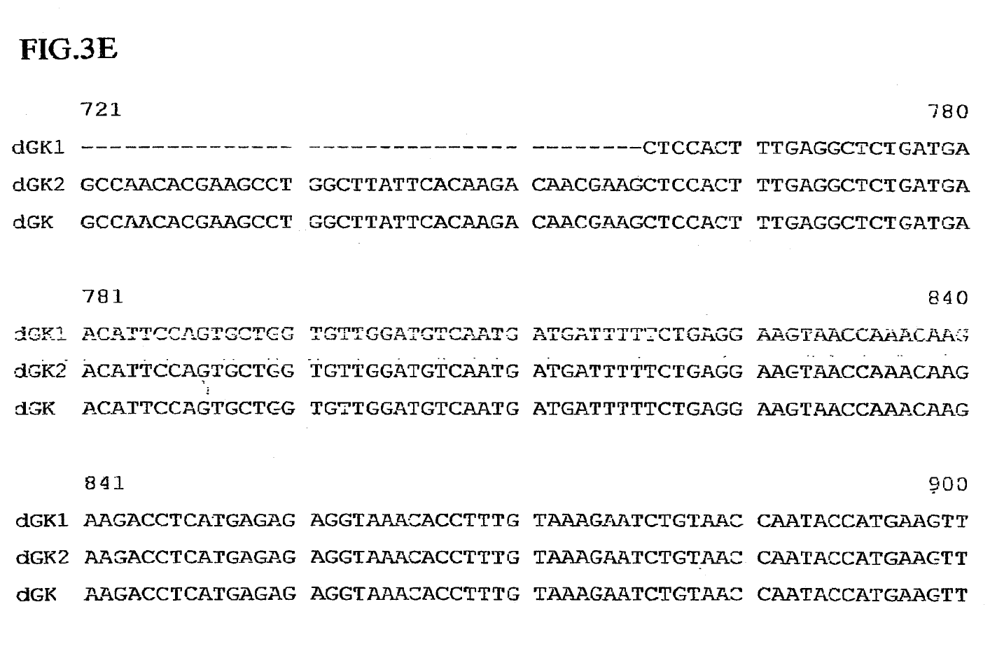

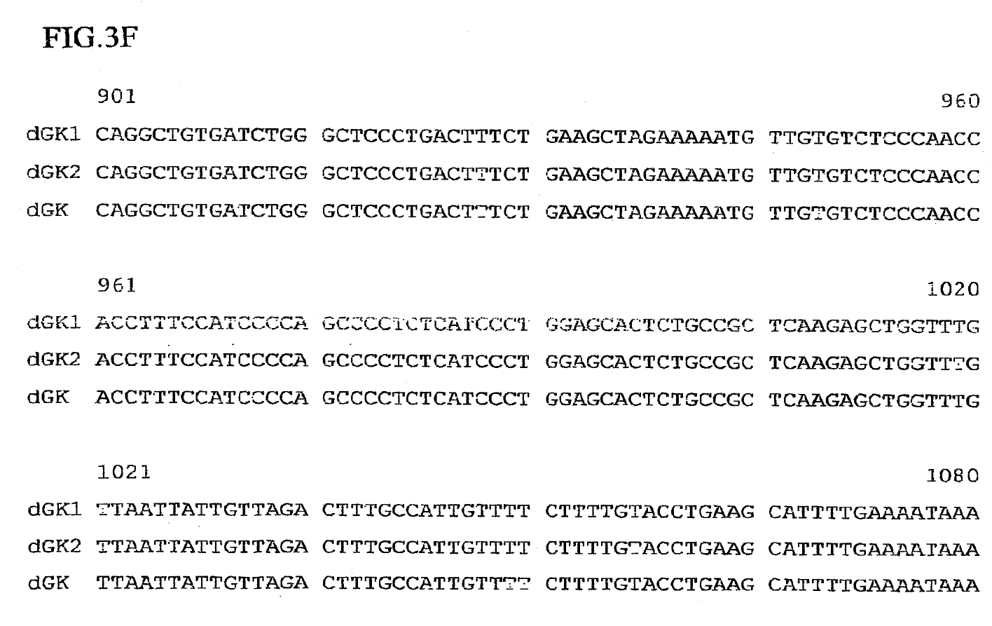

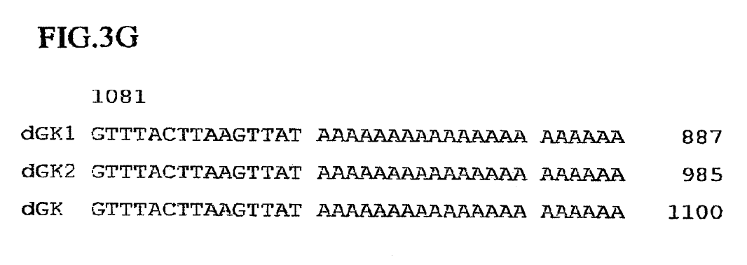

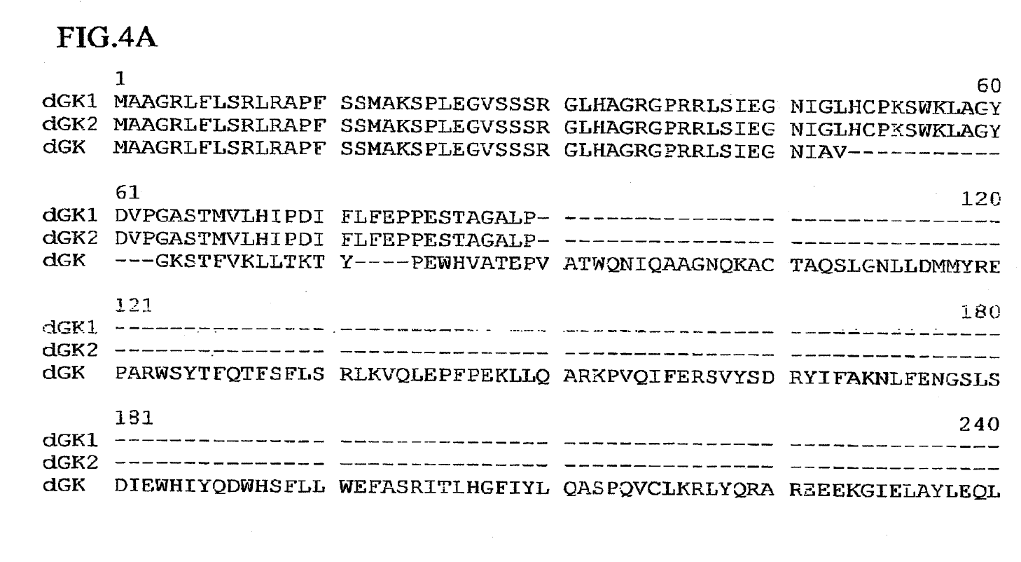

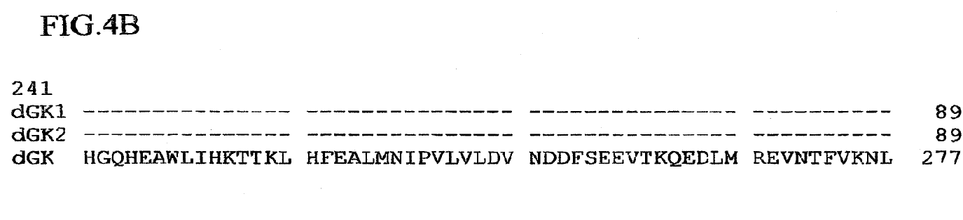

To further determine whether dGK1 and dGK2 are gene variants of dGK, alignments were performed to compare the sequences of dGK, dGK1 and dGK2 in nucleotides and amino acids (FIGS. 3A-G and 4A-B). The alignments showed that the nucleotide sequences of dGK1 and dGK2 are identical to dGK except for lacking some regions present in dGK. A 113 bp and a 116 bp of the sequence of dGK from nucleotides 177 to 289 and from nucleotides 627 to 742, respectively, are missing from dGK1. The missing regions are located between nucleotides 192 to 193 and 529 to 530 of dGK1, respectively. A 113 bp and a 18 bp of the sequence of dGK from nucleotides 177 to 289 and from nucleotides 462 to 479, respectively, are missing in dGK2. The missing regions are located between nucleotides 192 to 193 and 364 to 365 of dGK2, respectively. A previous study has reported the identification five isoforms of dGK (Johansson and Karlsson, (1996) Proc Natl Acad Sci U S A 93:7258-62). The ORF lengths of each of the five isoforms were 780 bp, 516 bp, 138 bp and two 216 bp. The authors also mentioned that these isoforms were formed by the presence or absence of the regions of sequence A (nucleotides 177 to 289 of dGK) or sequence B (nucleotides 479 to 742 of dGK). One of the isoform was formed in a different way by addition of an unrelated sequence replacing the region of sequence A. Both gene variants (dGK1 and dGK2) of the present invention were each formed by the absence of the sequence A and an additional region (116 bp and 18 bp, respectively). The 16 bp fragment absent in dGK1 is located in the region of sequence B, but shorter than sequence B in length. The 18 bp fragment absent in dGK2 is not located in either sequence A or sequence B. Both dGK1 and dGK2 encode the same predicted amino acid sequences. This is because the lacking of region A (113 bp) causes a frame-shift in the peptide sequence, which in turn generates a stop codon (corresponding to the amino acid position of 90 of dGK) upstream of their second genetic variations. Thus, both dGK1 and dGK2 encode C-terminally truncated polypeptides of dGK.

The functional domains of dGK have been classified into three regions (Ma et al. (1995) J Biol Chem 270:6595-601; Wang et al. (1996) FEBS Lett 390:39-43). These regions are: 1) the N-terminal part containing a mitochondrial leader sequence for entry into mitochondria (Gavel and von Heijne, (1990) Protein Eng 4:33-7; Nakai and Kanehisa, (1992) Genomics 14:897-911) and a glycine-rich region for ATP binding (Walker et al. (1982) EMBO J 1:945-51); 2) the middle part containing a DRS domain found in many viral thymidine kinases (Balasubramaniam et al. (1990) J Gen Virol 71:2979-87; Gentry, (1992) Pharmacol Ther 54:319-55); and 3) the C-terminal part containing arginine-rich domain for ATP phosphate binding (Ma et al. (1995) J Biol Chem 270:6595-601). A motif analysis indicates that only the N-terminal domains are conserved in dGK1 and dGK2 suggesting that both dGK1 and dGK2 may be functioning inside mitochondria.

To determine the tissue distribution of dGK1 and dGK2, an in silico Northern analysis was performed using a search of ESTs (originated from different cancer cell types) deposited in dbEST (Boguski et al., (1993) Nat Genet. 4: 332-3) at the National Center for Biotechnology Information (NCBI). Two ESTs were found to match the sequences of dGK1 and dGK2. The one (GenBank Accession No. BE392260) that matched to dGK1 was isolated from a cDNA library generated using uterus endometrium, adenocarcinoma cell line. The other one (GenBank Accession No. BE408107) that matched to dGK2 was isolated from a cDNA library generated using placenta choriocarcinoma. This result suggests that both dGK1 and dGK2 may be served as important indicators for cancers, more specifically, the uterus cancer and placenta cancer, respectively.

Therefore, the nucleotide fragments of dGAK1 of the invention comprising nucleotides 529 to 530, and the nucleotide fragments of dGAK2 of the invention comprising nucleotides 364 to 365 may be used as probes for determining the presence of the variants under highly stringent conditions. An alternative approach is that any set of primers for amplifying the fragment of dGAK1 comprising nucleotides 529 to 530 or the fragment of dGAK2 comprising nucleotides 364 to 365 may be used for determining the presence of the variants of the invention.

According to the present invention, the fragments of the nucleic acid sequences of the human dGK1 and dGK2 can be used as primers or probes. Preferably, the purified fragments of the human dGK1 and dGK2 are used. The fragments may be produced by enzyme digestion, chemical cleavage of isolated or purified nucleic acid sequences, or chemical synthesis and then may be isolated or purified. Such isolated or purified fragments of the nucleic acid sequences can be directly used as primers or probes.

Many gene variants have been found to be associated with diseases (Stallings-Mann et al., (1996) Proc Natl Acad Sci U S A 93: 12394-9; Liu et al., (1997) Nat Genet 16:328-9; Siffert et al., (1998) Nat Genet 18: 45-8; Lukas et al., (2001) Cancer Res 61: 3212 to 9). It is advisable that the dGK variants (dGK1 and dGK2) of the present invention, which have genetic deletion of nucleotide/amino acid sequences, may result in cancer development and be useful as markers for the diagnosis of human cancers. Based on the source of ESTs generated (cDNA libraries), the in silico tissue distribution analysis showed that dGK1 is associated with uterus cancer and dGK2 is associated with placenta cancer. Thus, the expression level of dGK1 or dGK21 relative to the expression level of dGK may be a useful indicator for screening of patients suspected of having uterus or placenta cancers, respectively. This suggests that the index of relative expression level (mRNA) may confer an increased susceptibility to uterus or placenta cancers. The fragments of dGK1 and dGK2 gene transcripts (mRNA) may be detected by RT-PCR approach. These approaches may be performed in accordance with conventional methods well known by persons skilled in the art.

The subject invention also provides methods for diagnosing the diseases associated with the deficiency of the dGK gene in a mammal, in particular, cancers, e.g. uterus cancer or placenta cancer.

The method for diagnosing the diseases associated with the deficiency of dGK gene may be performed by detecting the nucleotide sequences of the dGK1 and dGK2 variants of the invention which comprises the steps of: (1) extracting total RNA of cells obtained from the mammal; (2) amplifying the RNA by reverse transcriptase-polymerase chain reaction (RT-PCR) with a set of primers to obtain a cDNA comprising the fragments comprising nucleotides 527 to 532 of SEQ ID NO: 1 or nucleotides 362 to 367 of SEQ ID NO: 3; and (3) detecting whether the cDNA sample is obtained. If necessary, the amount of the obtained cDNA sample may be detected.

In the above embodiment, one of the primers may be designed to have a sequence comprising the nucleotides 527 to 532 of SEQ ID NO: 1 or the nucleotides 362 to 367 of SEQ ID NO: 3, and the other may be designed to have a sequence complementary to the nucleotides of SEQ ID NO: 1 at any other locations downstream of nucleotide 532 or to have a sequence complementary to the nucleotides of SEQ ID NO: 3 at any other locations downstream of nucleotide 367. Alternatively, one of the primers may be designed to have a sequence complementary to the nucleotides of SEQ ID NO: 1 containing nucleotides 527 to 532 or to have a sequence complementary to the nucleotides of SEQ ID NO: 3 containing nucleotides 362 to 367, and the other may be designed to have a sequence comprising the nucleotides of SEQ ID NO: 1 at any other locations upstream of nucleotide 527 or to have a sequence comprising the nucleotides of SEQ ID NO: 3 at any other locations upstream of nucleotide 362. In this case, only dGK1 or dGK2 will be amplified. In this case, the length of the PCR fragment from dGK=will be 116 bp shorter than that from dGK, and that of the PCR fragment from dGK2 will be 18 bp shorter than that from dGK. Preferably, the primers of the invention contain 15 to 30 nucleotides.

Alternatively, one of the primers may be designed to have a sequence comprising the nucleotides of SEQ ID NO: 1 at any locations from nucleotides 193 to 529 or to have a sequence comprising the nucleotides of SEQ ID NO: 3 at any locations from nucleotides 193 to 364, and the other may be designed to have a sequence complementary to the nucleotides of SEQ ID NO: 1 downstream of nucleotide 530 or to have a sequence complementary to the nucleotides of SEQ ID NO: 3 downstream of nucleotide 365. Alternatively, one of the primers may be designed to have a sequence complementary to the nucleotides of SEQ ID NO: 1 at any locations from nucleotides 193 to 529 or to have a sequence complementary to the nucleotides of SEQ ID NO: 3 at any locations from nucleotides 193 to 364, and the other may be designed to have a sequence comprising the nucleotides of SEQ ID NO: 1 downstream of nucleotide 530 or to have a sequence comprising the nucleotides of SEQ ID NO: 3 downstream of nucleotide 365. In this case, dGK1 or dGK2 together with dGK will be amplified. The length of the PCR fragment from dGK1 will be 116 bp shorter than that from dGK, and that of the PCR fragment from dGK2 will be 18 bp shorter than that from dGK.

Preferably, the primer of the invention contains 15 to 30 nulceotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected sizes of specific PCR fragments of gene variants (dGK1 and dGK2) can be used. PCR fragments are analyzed on a 1% agarose gel using five microliters (10%) of the amplified products. To determine the expression levels for each gene variants, the intensity of the PCR products may be determined by using the Molecular Analyst program (version 1.4.1; Bio-Rad).

The RT-PCR experiment may be performed according to the manufacturer instructions (Boehringer Mannheim). A 50 μl reaction mixture containing 2 μl total RNA (0.1 μg/μl), 1 μl each primer (20 pM), 1 μl each dNTP (10 mM), 2.5 μl DTT solution (100 mM), 10 μl 5×RT-PCR buffer, 1 μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment of the method for diagnosing the diseases associated with the deficiency of dGK gene is performed by detecting the of dGK1 or dGK2 variant of the invention which comprises the steps of: (1) extracting total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid selected from the group consisting of SEQ ID NOs: 1 and 3, and the fragments thereof; and (4) detecting whether the cDNA sample hybridizes with the nucleic acid of SEQ ID NO: 1 or 3, or the fragments thereof. If necessary, the amount of hybridized sample may be detected.

The expression of the gene variants can be analyzed using Northern Blot hybridization approach. Specific fragments, which comprises nucleotides 527 to 532 of SEQ ID NO: 1 or nucleotides 362 to 367 of SEQ ID NO: 3, may be amplified by polymerase chain reaction (PCR). The amplified PCR fragment may be labeled and serve as a probe to hybridize the membranes containing total RNAs extracted from the samples under the conditions of 55° C. in a suitable hybridization solution for 3 hr. Blots may be washed twice in 2×SSC, 0.1% SDS at room temperature for 15 minutes each, followed by two washes in 0.1×SSC and 0.1% SDS at 65° C. for 20 minutes each. After these washes, blot may be rinsed briefly in suitable washing buffer and incubated in blocking solution for 30 minutes, and then incubated in suitable antibody solution for 30 minutes. Blots may be washed in washing buffer for 30 minutes and equilibrated in suitable detection buffer before detecting the signals. Alternatively, the presence of the gene variant (cDNA or PCR) can be detected using microarray approach. The cDNAs or PCR products corresponding to the nucleotide sequences of the present invention may be immobilized on a suitable substrate such as a glass slide. Hybridization can be preformed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

The following examples are provided for illustration, but not for limiting the invention.

### EXAMPLES

### Analysis of Human Pooled Cancer Tissues EST Databases

Expressed sequence tags (ESTs) generated from the large-scale PCR-based sequencing of the 5′-end of pooled human cancer tissues cDNA clones were compiled and served as EST databases. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the NCBI with a significance cutoff of p<10⁻¹⁰. ESTs representing putative dGK encoding gene were identified during the course of EST generation.

### Isolation of cDNA Clones

Two cDNA clones exhibiting EST sequences similar to the dGK gene were isolated from a pooled cancer tissues cDNA library and named dGK1 and dGK2. The inserts of these clones were subsequently excised in vivo from the XZAP Express vector using the ExAssist/XLOLR helper phage system (Stratagene). Phagemid particles were excised by coinfecting XL1-BLUE MRF′ cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Epicentre#SE9101LC SequiTherm EXCEL™II DNA Sequencing Kit for 4200S-2 Global NEW IR² DNA sequencing system (LI-COR). Using the primer-walking approach, full-length sequence was determined. Nucleotide and protein searches were performed using BLAST against the non-redundant database of NCBI.

### In Silico Tissue Distribution Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variant with a significance cutoff of p<10⁻¹⁰.

### REFERENCES

Altschul et al., Gapped BLAST and PSI-BLAST: a new generation of protein database search programs, Nucleic Acids Res. 25: 3389-3402, (1997).

Arner and Eriksson. Mammalian deoxyribonucleoside kinases. Pharmacol Ther 67:155-86, (1995).

Balasubramaniam et al., Herpesviral deoxythymidine kinases contain a site analogous to the phosphoryl-binding arginine-rich region of porcine adenylate kinase; comparison of secondary structure predictions and conservation. J Gen Virol. 71:2979-87, (1990).

Boguski et al., dbEST—database for “expressed sequence tags”. Nat Genet. 4: 332-3, (1993).

Feder et al., Clinical relevance of chromosome abnormalities in non-small cell lung cancer. Cancer Genet Cytogenet. 102:25-31 (1998).

Gavel and von Heijne. Cleavage-site motifs in mitochondrial targeting peptides. Protein Eng. 4:33-7 (1990).

Gentry G. A. Viral thymidine kinases and their relatives. Pharmacol Ther. 54:319-55 (1992).

Gish and States. Identification of protein coding regions by database similarity search. Nat Genet 3:266-72 (1993).

Hengstschlager et al., A common regulation of genes encoding enzymes of the deoxynucleotide metabolism is lost after neoplastic transformation. Cell Growth Differ. 5:1389-94 (1994).

Hengstschlager et al., Different regulation of thymidine kinase during the cell cycle of normal versus DNA tumor virus-transformed cells. J Biol Chem. 269:13836-42 (1994).

Johansson and Karlsson. Cloning and expression of human deoxyguanosine kinase cDNA. Proc Natl Acad Sci U S A. 93:7258-62 (1996).

Johansson et al., Localization of the human deoxyguanosine kinase gene (DGUOK) to chromosome 2p13. Genomics 38:450-1 (1996).

Jones et al., Molecular cytogenetic comparison of apocrine hyperplasia and apocrine carcinoma of the breast. Am J Pathol. 158:207-14 (2001).

Jullig and Eriksson, Apoptosis induces efflux of the mitochondrial matrix enzyme deoxyguanosine kinase. J Biol Chem, 276:24000-4, (2001)

Kozak, An analysis of 5′-noncoding sequences from 699 vertebrate messenger RNAs. Nucleic Acids Res, 15: 8125-48, (1987).

Kozak, An analysis of vertebrate mRNA sequences: intimations of translational control, J Cell Biol, 115: 887-903, (1991).

Liu et al., Silent mutation induces exon skipping of fibrillin-1 gene in Marfan syndrome. Nat Genet 16:328-9, (1997).

Lui et al., High level amplification of 1p32-33 and 2p22-24 in small cell lung carcinomas. Int J Oncol. 19:451-7 (2001).

Lukas et al., Alternative and aberrant messenger RNA splicing of the mdm2 oncogene in invasive breast cancer. Cancer Res. 61:3212-9, (2001).

Ma et al., Cloning and expression of the heterodimeric deoxyguanosine kinase/deoxyadenosine kinase of Lactobacillus acidophilus R-26. J Biol Chem. 270:6595-601 (1995).

Merlo et al., Frequent microsatellite instability in primary small cell lung cancer. Cancer Res. 54:2098-101 (1994).

Nakai and Kanehisa. A knowledge base for predicting protein localization sites in eukaryotic cells. Genomics. 14:897-911 (1992).

Oliver et al., dNTP pools imbalance as a signal to initiate apoptosis. Experientia. 52:995-1000 (1996).

Otsuka et al., Deletion mapping of chromosome 2 in human lung carcinoma. Genes Chromosomes Cancer. 16:113-9 (1996).

Reed, Dysregulation of apoptosis in cancer. J Clin Oncol, 17:2941-53, (1999).

Siffert et al., Association of a human G-protein beta3 subunit variant with hypertension. Nat Genet. 18:45-8, (1998).

Sjostrom and Bergh, How apoptosis is regulated, and what goes wrong in cancer. BMJ, 322:1538-9, (2001).

Stallings-Mann et al., Alternative splicing of exon 3 of the human growth hormone receptor is the result of an unusual genetic polymorphism. Proc Natl Acad Sci U S A. 93:12394-9, (1996).

Strausberg R. EST Accession No. BE392260 and BE408107

Summersgill et al., Chromosomal imbalances associated with carcinoma in situ and associated testicular germ cell tumours of adolescents and adults. Br J Cancer. 85:213-20 (2001).

Tattersall et al., Deoxyribonucleoside triphosphate pools in human bone marrow and leukaemic cells. Antibiot Chemother, 28:94-101, (1980).

Walker et al., Distantly related sequences in the alpha- and beta-subunits of ATP synthase, myosin, kinases and other ATP-requiring enzymes and a common nucleotide binding fold. EMBO J. 1:945-51 (1982).

Wang et al., Cloning and expression of human mitochondrial deoxyguanosine kinase cDNA. FEBS Lett. 390:39-43 (1996).

Willatt et al., Partial trisomy of 2p and neuroblastoma. Am J Med Genet. 102:304-5 (2001).

Wyllie et al., Apoptosis and carcinogenesis. Br J Cancer, 80 Suppl 1:34-7, (1999).

Zornig et al, Apoptosis regulators and their role in tumorigenesis. Biochim Biophys Acta. 1551:F1-37 (2001).

What is claimed is:

1. An isolated nucleic acid comprising the nucleotide sequence of SEQ ID NO: 1 or 3, and fragments thereof.

2. The isolated nucleic acid of claim 1, wherein the fragments comprise the nucleotides 527 to 532 of SEQ ID NO: 1.

3. The isolated nucleic acid of claim 1, wherein the fragments comprise the nucleotides 362 to 367 of SEQ ID NO: 3.

4. A method for diagnosing diseases associated with the deficiency of dGK gene, in particular, cancers, in a mammal which comprises detecting the nucleic acid of any one of claims 1 to 3.

5. The method of claim 4, wherein the disease is uterus cancer or placenta cancer.

6. The method of claim 4, wherein the detection of the nucleic acid of any one of claims 1 to 3 comprising the steps of:
(1) extracting total RNA from a sample obtained from the mammal;
(2) amplifying the RNA by reverse transcriptase-polymerase chain reaction (RT-PCR) with a pair of primers to obtain a cDNA sample comprising the nucleotides 527 to 532 of SEQ ID NO: 1 or nucleotides 362 to 367 of SEQ ID NO: 3; and
(3) detecting whether the cDNA sample is obtained.

7. The method of claim 6, wherein one of the primers has a sequence comprising the nucleotides 527 to 532 of SEQ ID NO: 1 or the nucleotides 362 to 367 of SEQ ID NO: 3, and the other has a sequence complementary to the nucleotides of SEQ ID NO: 1 at any other locations downstream of nucleotide 532 or has a sequence complementary to the nucleotides of SEQ ID NO: 3 at any other locations downstream of nucleotide 367, or one of the primers has a sequence complementary to the nucleotides of SEQ ID NO: 1 containing nucleotides 527 to 532 or has a sequence complementary to the nucleotides of SEQ ID NO: 3 containing nucleotides 362 to 367, and the other has a sequence comprising the nucleotides of SEQ ID NO: 1 at any other locations upstream of nucleotide 527 or has a sequence comprising the nucleotides of SEQ ID NO: 3 at any other locations upstream of nucleotide 362.

8. The method of claim 6, wherein one of the primers has a sequence comprising the nucleotides of SEQ ID NO: 1 at any locations from nucleotides 193 to 529 or has a sequence comprising the nucleotides is of SEQ ID NO: 3 at any locations from nucleotides 193 to 364, and the other has a sequence complementary to the nucleotides of SEQ ID NO: 1 downstream of nucleotide 530 or has a sequence complementary to the nucleotides of SEQ ID NO: 3 downstream of nucleotide 365, or one of the primers has a sequence complementary to the nucleotides of SEQ ID NO: 1 at any locations from nucleotides 193 to 529 or has a sequence complementary to the nucleotides of SEQ ID NO: 3 at any locations from nucleotides 193 to 364, and the other has a sequence comprising the nucleotides of SEQ ID NO: 1 downstream of nucleotide 530 or to have a sequence comprising the nucleotides of SEQ ID NO: 3 downstream of nucleotide 365.

9. The method of claim 8, the cDNA sample amplified from SEQ ID NO: 1 is 116 bp shorter than that from dGK.

10. The method of claim 8, the cDNA sample amplified from SEQ ID NO: 3 is 18 bp shorter than that from dGK.

11. The method of claim 6 further comprising the step of detecting the amount of the amplified cDNA sample.

12. The method of claim 4, wherein the detection of the nucleic acid of any one of claims 1 to 3 comprises the steps of:
(1) extracting the total RNA of a sample obtained from the mammal;
(2) amplifying the RNA by reverse transcriptase-polymerase chain reaction (RT-PCR) to obtain a cDNA sample;
(3) bringing the cDNA sample into contact with the nucleic acid of any one of claims 1 to 3; and
(4) detecting whether the cDNA sample hybridizes with the nucleic acid of any one of claims 1 to 3.

13. The method of claim 12 further comprising the step of detecting the amount of hybridized sample.


SEQUENCE LISTING (6 sequences)

SEQ ID NO: 1 - DNA, 887 bp, Homo sapiens
  CDS (51)..(317)
ttagcaggatacctagggcggaagtgatcgctgtgtgaatcgtgggtgggatggccgcgggccgcctctttctaagtcggcttcgagcacccttcagttccatggccaagagcccactcgagggcgtttcctcctccagaggcctgcacgcggggcgcgggccccgaaggctctccatcgaaggcaacattggcctgcactgcccaaagtcttggaaacttgctggatatgatgtaccgggagccagcacgatggtcctacacattccagacattttcctttttgagccgcctgaaagtacagctggagcccttccctgagaaactcttacaggccaggaagccagtacagatctttgagaggtctgtgtacagtgacaggtatatctttgcaaagaatctttttgaaaatggttccctcagtgacatcgagtggcatatctatcaggactggcattcttttctcctgtgggagtttgccagccggatcacattacatggcttcatctacctccaggcttctccccaggctccactttgaggctctgatgaacattccagtgctggtgttggatgtcaatgatgatttttctgaggaagtaaccaaacaagaagacctcatgagagaggtaaacacctttgtaaagaatctgtaaccaataccatgaagttcaggctgtgatctgggctccctgactttctgaagctagaaaaatgttgtgtctcccaaccacctttccatccccagcccctctcatccctggagcactctgccgctcaagagctggtttgttaattattgttagactttgccattgttttcttttgtacctgaagcattttgaaaataaagtttacttaagttataaaaaaaaaaaaaaaaaaaaa

SEQ ID NO: 2 - protein, 89 aa, Homo sapiens
MAAGRLFLSRLRAPFSSMAKSPLEGVSSSRGLHAGRGPRRLSIEGNIGLHCPKSWKLAGYDVPGASTMVLHIPDIFLFEPPESTAGALP

SEQ ID NO: 3 - protein, 89 aa, Homo sapiens; identical to SEQ ID NO: 2

SEQ ID NO: 4 - DNA, 985 bp, Homo sapiens
  CDS (57)..(317)
ttagcaggatacctagggcggaagtgatcgctgtgtgaatcgtgggtgggatggccgcgggccgcctctttctaagtcggcttcgagcacccttcagttccatggccaagagcccactcgagggcgtttcctcctccagaggcctgcacgcggggcgcgggccccgaaggctctccatcgaaggcaacattggcctgcactgcccaaagtcttggaaacttgctggatatgatgtaccgggagccagcacgatggtcctacacattccagacattttcctttttgagccgcctgaaagtacagctggagcccttccctgagaaactcttacaggccaggaagccagtacagatctttgagaggtatatctttgcaaagaatctttttgaaaatggttccctcagtgacatcgagtggcatatctatcaggactggcattcttttctcctgtgggagtttgccagccggatcacattacatggcttcatctacctccaggcttctccccaggtttgtttgaagagactgtaccagagggccagggaggaggagaaaggaattgagctggcctatctagagcagctgcatggccaacacgaagcctggcttattcacaagacaacgaagctccactttgaggctctgatgaacattccagtgctggtgttggatgtcaatgatgatttttctgaggaagtaaccaaacaagaagacctcatgagagaggtaaacacctttgtaaagaatctgtaaccaataccatgaagttcaggctgtgatctgggctccctgactttctgaagctagaaaaatgttgtgtctcccaaccacctttccatccccagcccctctcatccctggagcactctgccgctcaagagctggtttgttaattattgttagactttgccattgttttcttttgtacctgaagcattttgaaaataaagtttacttaagttataaaaaaaaaaaaaaaaaaaaa

SEQ ID NO: 5 - protein, 87 aa, Homo sapiens
AGRLFLSRLRAPFSSMAKSPLEGVSSSRGLHAGRGPRRLSIEGNIGLHCPKSWKLAGYDVPGASTMVLHIPDIFLFEPPESTAGALP

SEQ ID NO: 6 - protein, 89 aa, Homo sapiens; identical to SEQ ID NO: 2